SEQUENCE LISTING (3 sequences)

SEQ ID NO: 1 - DNA, 26 bp, Artificial Sequence
  Description of Artificial Sequence Synthetic oligonucleotide
gggacttggtttaggtaatgagtccc

SEQ ID NO: 2 - DNA, 45 bp, Artificial Sequence
  Description of Artificial Sequence Synthetic oligonucleotide
cgagggtaccgcaatagtacttattgttcgcctattgtgggtcgg

SEQ ID NO: 3 - DNA, 29 bp, Artificial Sequence
  Description of Artificial Sequence Synthetic oligonucleotide
gacaaggaaatccttcaacgaagtgggtc

(19) United States
(12) Patent Application Publication
(10) Pub. No.: US 2024/0125729 A1
(43) Pub. Date: Apr. 18, 2024

(54) METHODS AND RELATED ASPECTS OF DETECTING TARGET MOLECULES USING CYCLIC VOLTAMMETRY

(71) Applicant: THE JOHNS HOPKINS UNIVERSITY, Baltimore, MD (US)

(72) Inventors: Netzahualcoyotl ARROYO, Baltimore, MD (US); Miguel Aller PELLITERO, Baltimore, MD (US); Jonathan D. MAHLUM, Baltimore, MD (US); Samuel D. CURTIS, Baltimore, MD (US)

(73) Assignee: THE JOHNS HOPKINS UNIVERSITY, Baltimore, MD (US)

(21) Appl. No.: 18/277,258

(22) PCT Filed: Feb. 18, 2022
(86) PCT No.: PCT/US2022/017102

§ 371 Date: Aug. 15, 2023

Related U.S. Application Data
(60) Provisional application No. 63/151,478, filed on Feb. 19, 2021.

(51) Int. Cl. G01N 27/327; A61B 5/00; A61B 5/1477; G01N 27/48; G01N 33/543

(52) U.S. Cl. CPC G01N 27/3277; A61B 5/1477; A61B 5/685; G01N 27/3276; G01N 27/48; G01N 33/5438

(57) ABSTRACT

Provided herein are methods of detecting target molecules using electrochemical sensors that comprise biomolecular receptor-bound redox reporters. Related systems and computer readable media are also provided.

### CROSS-REFERENCE TO RELATED APPLICATIONS

This application is the national stage entry of International Patent Application No. PCT/US2022/017102, filed on Feb. 18, 2022, and published as WO 2022/178327 A1 on Aug. 25, 2022, which claims the benefit of U.S. Provisional Patent Application Ser. No. 63/151,478, filed Feb. 19, 2021, both of which are hereby incorporated by reference herein in their entireties.

### SEQUENCE LISTING

The instant application contains a Sequence Listing which has been submitted electronically in ASCII format and is hereby incorporated by reference in its entirety. Said ASCII copy, created on Feb. 17, 2022, is named 0184_0125-PCT_SL.txt and is 1,012 bytes in size.

### BACKGROUND

Electrochemical, aptamer-based (E-AB) sensors are analytical platforms that achieve continuous monitoring of specific molecular targets in vivo. E-AB sensors present an architecture typically consisting of three elements (FIG. 1A): 1) a self-assembled monolayer (SAM) of target-binding, alkanethiol-functionalized nucleic-acid aptamers or other bioreceptor, 2) an electrode-blocking SAM of alkanethiols to prevent undesired electrochemical reactions and confer biocompatibility to the electrode surface, and 3) a redox reporter sensitive to target-binding events. The redox reporter, typically methylene blue (MB), is attached to the terminal end of the aptamer, opposite to the electrode attachment terminus. In the presence of target, aptamer molecules reversibly undergo binding-induced conformational changes that presumably bring the reporter closer to the electrode surface, causing a change in the electron transfer rate between the reporter and the electrode (FIG. 1B), which can be easily measured electrochemically. Aptamer binding in E-AB sensors is at dynamic equilibrium, reversibly switching between bound and unbound states at rates of milliseconds. This behavior makes E-AB sensors ideal for continuous monitoring applications. Moreover, because their working principle mimics the binding-induced conformational changes seen in naturally occurring chemoreceptors in the body, E-AB sensors tolerate prolonged measurements in complex matrices such as unprocessed biological fluids.

E-AB sensors can be successfully interrogated via chronoamperometry, differential pulse techniques such as square-wave voltammetry and differential pulse voltammetry, alternating current voltammetry, and electrochemical impedance spectroscopy. Ultimately, the choice of technique is typically determined by the final intended application of the E-AB sensor. For example, the simplicity of the voltage program in chronoamperometry is ideal for drift-free measurements at sub-second interrogation frequencies, which may be needed for the study of fast biological processes like neurotransmitter modulation in the brain. Electrochemical impedance, in contrast, offers the convenience of interrogating E-AB sensors in a label-free manner, without using a redox reporter. However, the vast majority of reported E-AB sensors have been interrogated by pulse techniques and, in particular, by square wave voltammetry. This widespread use likely arose because pulsed techniques differentially remove currents originating from charging the electrode-electrolyte double layer, significantly improving the signal-to-noise ratio of E-AB measurements. Yet, pulsed techniques also remove valuable electrochemical information regarding sensor stability (e.g., the capacitive current reports on monolayer stability) and differential voltage pulsing also strains the E-AB interface causing faster loss of signal.

Cyclic voltammetry (CV) is frequently used for the surface characterization of E-AB sensors, as this technique provides valuable information regarding monolayer stability (by proxy of double layer capacitance) and surface coverage of the redox reporter-modified aptamer (from faradaic peak areas). However, CV is not commonly used for the direct interrogation of E-AB sensors, in part because sensors with defective blocking monolayers or redox reporter-modified aptamers with slow electron transfer kinetics present large capacitive currents that can hide the faradaic waves of methylene blue, resulting in low signal-to-noise E-AB measurements. Moreover, for many E-AB sensors, CV peak currents do not change significantly with increasing target concentrations.

Accordingly, there is a need for additional methods, and related aspects, of interrogating electrochemical sensors using cyclic voltammetry.

### SUMMARY

The present disclosure relates, in certain aspects, to methods, systems, and computer readable media of use in detecting target molecules using cyclic voltammetry (CV). Some aspects, for example, include a general CV-based interrogation approach that employs voltammogram peak-to-peak separation (ΔE_{P}) to directly measure binding-induced changes in the apparent electron transfer kinetics of E-AB sensors. Certain embodiments demonstrate that voltage programs used in CV are less damaging to E-AB interfaces over time, and that ΔE_{P}-based interrogation can achieve E-AB measurements with significantly reduced batch-to-batch and day-to-day variability relative to the benchmark square wave voltammetry. These and other aspects will be apparent upon a complete review of the present disclosure, including the accompanying figures.

In one aspect, the present disclosure provides a method of detecting a target molecule using an electrochemical sensor comprising biomolecular receptor-bound redox reporters. The method includes contacting the electrochemical sensor with at least one sample that comprises the target molecule such that one or more of the biomolecular receptors undergo conformational changes when the biomolecular receptors bind the target molecule. The method also includes generating one or more cyclic voltammograms from the electrochemical sensor using cyclic voltammetry (CV). In addition, the method also includes determining a change in a target peak-to-peak separation, ΔE_{P,T}, from the cyclic voltammograms generated from the electrochemical sensor, thereby detecting the target molecule using the electrochemical sensor. In some embodiments, electrochemical sensors are configured to be worn by users as wearable devices (e.g., wearable microneedle sensor arrays, etc.) that continuously detect and monitor target molecule concentrations in and/or on the users.

In some embodiments, the determining step comprises comparing the ΔE_{P,T} to a no target peak-to-peak separation, ΔE_{P,NT}, determined from one or more cyclic voltammograms generated from the electrochemical sensor in the absence of the target molecule. In some embodiments, the methods disclosed herein include determining a concentration of the target molecule in the sample by comparing the ΔE_{P,T} to a standard curve. In some embodiments, the methods disclosed herein include determining the ΔE_{P,T} from at least a first cyclic voltammogram and at least a second cyclic voltammogram generated from the electrochemical sensor. In some embodiments, the determining step comprises correlating at least two currents with corresponding peak potentials and calculating a separation between the peak potentials.

In some embodiments, the methods disclosed herein include determining a concentration of the target molecule in the sample via the change in the target peak-to-peak separation, ΔE_{P,T}. In some embodiments, the electrochemical sensor is substantially resistant to drift. In some embodiments, the methods disclosed herein include determining the change in the target peak-to-peak separation, ΔE_{P,T}, from the cyclic voltammograms with about 900 milliseconds, about 800 milliseconds, about 700 milliseconds, about 600 milliseconds, about 500 milliseconds, about 400 milliseconds, about 300 milliseconds, about 200 milliseconds, about 100 milliseconds, or less of contacting the electrochemical sensor with the sample. In some embodiments, the methods disclosed herein include generating the cyclic voltammograms from the electrochemical sensor using a voltage scanning rate of about 5 V s⁻¹ or more. In some embodiments, the voltage scanning rate is between about 5 V s⁻¹ and about 10 V s⁻¹. In some embodiments, the methods disclosed herein include continuously monitoring the change in the target peak-to-peak separation, ΔE_{P,T} over time from multiple cyclic voltammograms generated from the electrochemical sensor.

In some embodiments, the biomolecular receptor comprises an aptamer. In some embodiments, the biomolecular receptor comprises a deoxyribonucleic acid (DNA) molecule. In some embodiments, the redox reporters comprise methylene blue (MB).

In some embodiments, the sample is substantially unprocessed. In some embodiments, the sample comprises an environmental sample. In some embodiments, the target molecule comprises a therapeutic agent. In some embodiments, the methods disclosed herein include generating a dose-response curve for the therapeutic agent. In some embodiments, the target molecule comprises a metabolite. In some embodiments, the target molecule comprises a biomarker (e.g., a biomolecule or the like).

In some embodiments, the sample comprises a biological sample. In some embodiments, the biological sample is obtained from a subject. In some embodiments, the biological sample is selected from the group consisting of: serum, plasma, blood, saliva, interstitial fluid, urine, feces, semen, and cerebrospinal fluid.

In another aspect, the present disclosure provides a system that includes at least one electrochemical sensor comprising biomolecular receptor-bound redox reporters. The system also includes at least one controller operably connected to the electrochemical sensor. The controller comprises, or is capable of accessing, computer readable media comprising non-transitory computer executable instructions which, when executed by at least one electronic processor, perform at least: generating one or more cyclic voltammograms from the electrochemical sensor using cyclic voltammetry (CV) when the electrochemical sensor is contacted with at least one sample that comprises the target molecule such that one or more of the biomolecular receptors undergo conformational changes when the biomolecular receptors bind the target molecule; and determining a change in a target peak-to-peak separation, ΔE_{P,T}, from the cyclic voltammograms generated from the electrochemical sensor to detect the target molecule in the sample.

In another aspect, the present disclosure provides computer readable media comprising non-transitory computer executable instructions which, when executed by at least electronic processor, perform at least: generating one or more cyclic voltammograms from an electrochemical sensor comprising biomolecular receptor-bound redox reporters using cyclic voltammetry (CV) when the electrochemical sensor is contacted with at least one sample that comprises the target molecule such that one or more of the biomolecular receptors undergo conformational changes when the biomolecular receptors bind the target molecule; and determining a change in a target peak-to-peak separation, ΔE_{P,T}, from the cyclic voltammograms generated from the electrochemical sensor to detect the target molecule in the sample.

In some embodiments of the system or computer readable media disclosed herein, the instructions further perform at least: comparing the ΔE_{P,T} to a no target peak-to-peak separation, ΔE_{P,NT}, determined from one or more cyclic voltammograms generated from the electrochemical sensor in the absence of the target molecule. In some embodiments of the system or computer readable media disclosed herein, the instructions further perform at least: determining a concentration of the target molecule in the sample by comparing the ΔE_{P,T} to a standard curve. In some embodiments of the system or computer readable media disclosed herein, the instructions further perform at least: determining the ΔE_{P,T} from at least a first cyclic voltammogram and at least a second cyclic voltammogram generated from the electrochemical sensor. In some embodiments of the system or computer readable media disclosed herein, the instructions further perform at least: determining a concentration of the target molecule in the sample via the change in the target peak-to-peak separation, ΔE_{P,T}. In some embodiments of the system or computer readable media disclosed herein, the electrochemical sensor comprises a wearable device that is worn by the subject.

In some embodiments of the system or computer readable media disclosed herein, the electrochemical sensor is substantially resistant to drift. In some embodiments of the system or computer readable media disclosed herein, the cyclic voltammograms are determined from the electrochemical sensor using a voltage scanning rate of about 5 V s⁻¹ or more. In some embodiments of the system or computer readable media disclosed herein, the target molecule comprises a therapeutic agent and wherein the instructions further perform at least: generating a dose-response curve for the therapeutic agent. In some embodiments of the system or computer readable media disclosed herein, the instructions further perform at least: continuously monitoring the change in the target peak-to-peak separation, ΔE_{P,T} over time from multiple cyclic voltammograms generated from the electrochemical sensor. In some embodiments of the system or computer readable media disclosed herein, the biomolecular receptor comprises an aptamer. In some embodiments of the system or computer readable media disclosed herein, the biomolecular receptor comprises a deoxyribonucleic acid (DNA) molecule. In some embodiments of the system or computer readable media disclosed herein, the redox reporters comprise methylene blue (MB).

In some embodiments of the system or computer readable media disclosed herein, the biomolecular receptor comprises an aptamer. In some embodiments of the system or computer readable media disclosed herein, the biomolecular receptor comprises a deoxyribonucleic acid (DNA) molecule. In some embodiments of the system or computer readable media disclosed herein, the DNA molecule is single-stranded. In some embodiments of the system or computer readable media disclosed herein, the DNA molecule is double-stranded. In some embodiments of the system or computer readable media disclosed herein, the redox reporters comprise methylene blue (MB).

### BRIEF DESCRIPTION OF THE DRAWINGS

The accompanying drawings, which are incorporated in and constitute a part of this specification, illustrate certain embodiments, and together with the written description, serve to explain certain principles of the methods, systems, and related computer readable media disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 (panels A and B) schematically show that E-AB sensors undergo target binding-induced changes in electron transfer kinetics of the redox reporter that can be monitored in real time via electrochemical interrogation according to one exemplary embodiment. (A) In this work we employed three different DNA aptamers modified at the 5′ terminus with alkanethiol linkers and at the 3′ terminus with the redox reporter methylene blue. We co-deposited these modified oligonucleotides with 6-mercapto-1-hexanol on the surface of gold electrodes via self-assembly. (B) In the presence of their target molecule, the aptamers undergo a conformational change that, presumably, brings the redox reporter closer to the electrode surface, increasing the electron transfer rate.

FIG. 2 is a flow chart that schematically depicts steps in a method of detecting a target molecule using an electrochemical sensor comprising biomolecular receptor-bound redox reporters according to an exemplary embodiment.

FIG. 3 shows an algorithm according to an exemplary embodiment.

FIG. 4 schematically depicts a system according to an exemplary embodiment.

FIG. 5 (panels A-C) are plots showing target binding-induced changes in apparent electron transfer rates of E-AB sensors can be monitored via ΔE_{P}. (A) When we interrogate tobramycin-detecting E-AB sensors using cyclic voltammetry (at 5 V·s⁻¹), we observe a decrease in ΔE_{P} upon addition of tobramycin (10 mM). (B) Same voltammograms as in (A) but zoomed-in relative to x-axis show more clearly the change in electron transfer rate (kₑₜ) from ΔE_{P,1} to ΔE_{P,2}, with ΔE_{P,2}<ΔE_{P,1}. (C) The magnitude of ΔE_{P} decreases monotonically with increasing target concentration. The solid line is a non-linear fit to the Hill equation, resulting in a Hill coefficient n=1 and K_{D}=80±7 μM. Error bars represent the standard deviation between 5 electrodes. All measurements performed in lx phosphate-buffered saline.

FIG. 6 are plots showing the effect of CV scan rate on the shape of E-AB sensor dose-response curve. Here we illustrate this effect using tobramycin-binding E-AB sensors in phosphate-buffered saline for scan rates ranging from 0.5 to 100 V·s⁻¹. Solid lines correspond to non-linear regression analyses using the Hill isotherm (at fixed n=1). Error bars represent the standard deviation between 5 sensors.

FIG. 7 (panels A-C) are plots showing E-AB sensor gain and sensitivity based on ΔE_{P} are a strong function of voltage scanning rate. (A) We illustrate this effect by showing ΔE_{P} values in the absence (black circles, ΔE_{P,1}) and presence (red circles, ΔE_{P,2}) of saturating concentrations of tobramycin (10 mM) at increasing voltage scanning rates. Note that, although ΔE_{P} increases with increasing scanning rate in both cases, it does not do so with the same correlation function. Thus, CV scanning rates ranging between 5 V·s⁻¹ and 10 V·s⁻¹ produce the largest signal change based on ΔE_{P}. (B) Dose-response curves built from ΔE_{P} measurements result in apparent aptamer dissociation constants that strongly depend on CV scanning rate. Interrogating tobramycin-detecting E-AB sensors at CV scanning rates between 5 V·s⁻¹ and 10 V·s⁻¹ achieve the most sensitive measurements (lowest K_{D}) with the largest overall signal gain. (C) Side-by-side comparison of sensors interrogated by CV using voltammetric peak heights, I_{P} (blue circles) vs ΔE_{P} (black circles), to illustrate that ΔE_{P} achieves a 3-fold improvement in signal gain relative to I_{P}, albeit with opposite sign. Error bars represent the standard deviation between 5 electrodes. All measurements performed in 1× phosphate-buffered saline.

FIG. 8 are plots showing peak broadening at fast voltage scanning rates. The broadening observed at voltage scanning rates faster than 10 V·s can hinder the accurate readout of peak potential.

FIG. 9 is a plot showing a comparison of signal change using I_{P} or ΔE_{P} as analytical parameters. The increase in electron transfer rate between MB and the electrode surface upon tobramycin addition (10 mM) is reflected by a decrease in peak-to-peak separation of cyclic voltammograms (ΔE_{P,1} and ΔE_{P,2} for the unbound and bound states, respectively). However, this variation in electron transfer rate is minimally reflected as a change in peak current, observing only a slight increase at high scan rates (I_{P,1} and I_{P,2} for the unbound and bound states, respectively). Error bars represent the standard deviation between 5 sensors.

FIG. 10 (panels A and B) are plots that show changes in I_{P} and ΔE_{P} at slow rates. (A) The interrogation of tobramycin-binding E-AB sensors at a low scan rate (1 V·s⁻¹) leads to no changes in peak current upon addition of a saturating tobramycin concentrations (10 mM). (B) Thus, the resulting titration curve is flat when I_{P} is used as the analytical parameter. In contrast, when we use ΔE_{P}, a sigmoidal titration curve is obtained, reflecting the suitability of the ΔE_{P}-based method to interrogate E-AB sensors even at such slow voltage scanning rates. Error bars represent the standard deviation between 5 electrodes.

FIG. 11 (panels A-C) are plots that show ΔE_{P}-based interrogation supports E-AB measurements irrespective of the aptamer used. We evaluated the general suitability of our method for E-AB sensing in undiluted serum by fabricating sensors using (A) tobramycin-, (B) vancomycin-, and (C) procaine-binding aptamers. Here we show the performance of these sensors in undiluted serum when interrogated by CV. Left. Cyclic voltammograms measured in the absence (black trace) and presence (red trace) of saturating target show significant differences in ΔE_{P}. Note that for tobramycin and vancomycin we observed minimal change in I_{P}. However, voltammograms measured with the procaine sensor did show a significant change in I_{P} matching a broadening of the faradaic waves. This wave broadening is related to the protonation of a radical methylene blue intermediate, which we discuss further herein. Center. Scan rate dependency of each sensor to determine the region of maximum signal decrease between the unbound and bound states. Right. We use the scanning rate giving the best compromise between maximum signal change and low apparent K_{D} to build calibration curves for each analyte. Error bars represent the standard deviation between 5 electrodes.

FIG. 12 is a plot showing a calibration curve for procaine-binding E-AB sensors obtained by square wave voltammetry. The Hill's coefficient and dissociation constant obtained were 0.97±0.05 and 1.9±0.2 mM, respectively. Each point represents the average of 5·measurements at a frequency of 100 Hz, amplitude of 50 mV, and step potential of 1 mV.

FIG. 13 (panels A-C) are plots that show serially interrogating E-AB sensors via ΔE_{P} supports second to sub-second monitoring of fluctuating target concentrations in real time. Here we show serial ΔE_{P} measurements (black traces) recorded at (A) 5 V·s⁻¹ for tobramycin, (B) 1 V·s⁻¹ for vancomycin, and (C) 1 V·s⁻¹ for procaine. Using a voltage window of 600 mV (e.g., see FIG. 5A), these scanning rates achieve measurements every 0.24 s, 1.2 s and 1.2 s, respectively. To demonstrate E-AB performance over time, in these panels we recorded measurements for 7 hours alternating between 100% serum, and serum+target at a saturating concentration. To reveal the percentage contribution of drift to our measurements, we present the data as relative change in signal with respect to the signal measured at the start of each experiment. We eliminated the effect of temperature fluctuations by maintaining the electrochemical cell at 25° C. using a water jacket and a temperature-controlled recirculation bath. We also continuously stirred the serum at ˜100 rpms to avoid precipitation of solids from serum. Red traces represent the expected relative signal change in the absence or presence of target as given by our calibration curves from FIG. 11. Error bars represent the standard deviation between 5 electrodes.

FIG. 14 (panels A and B) are plots that show long-term stability of E-AB sensors under different interrogation methods. (A) To investigate the extent to which continuously interrogating the sensors affects their operational stability, we interrogated E-AB sensors in phosphate-buffered saline for 72 h by square-wave voltammetry, either in continuous (5·10⁴ scans) or single-point (10 scans) regimes, and by CV (7·10⁴ scans) monitoring both peak-to-peak separation and the oxidation peak current. We observe that continuous square-wave voltammetry-based interrogation contributes to a fast loss in signal (red trace), which dramatically decreases with less total measurements (at equal total experiment time, green circles). However, continuous CV-based interrogation (7·10⁴ scans) by monitoring either ΔE_{P} or IP does not contribute to sensor signal loss. (B) When we switched to a more complex matrix like undiluted serum, CV-based interrogation presents an initial loss of signal (˜15%) during the first 10 h likely due to monolayer reorganization, desorption, and the non-specific binding of proteins. While CV peak currents continue to drop linearly after this initial decay (blue trace), ΔE_{P} remains constant for ˜50 h, the point at which our software is no longer able to resolve voltammetric peaks from charging current. These measurements were performed using tobramycin-binding E-AB sensors at controlled 25° C. and under continuous stirring. Shaded areas or error bars represent the standard deviation between 5 electrodes.

FIG. 15 (panels A and B) are plots that show the batch-to-batch and day-to-day performance of E-AB sensors in undiluted serum is affected by the sensitivity of the interrogation method to changing sensor interfaces. (A) Square-wave voltammetry-based measurements of E-AB peak current performed continuously every 5 seconds for 24 hours. These measurements present significant variability in signal output between 3 batches of 6 sensors each (different colored traces), measured on 3 separate days. (B) The same variability is not seen for ΔEP-based interrogation, which produces indistinguishable traces between electrode batches and measurement days. Specifically, the initial decay in signal driven by monolayer reorganization and non-specific protein binding is identical between batches and, in all cases, it stabilizes at ˜75% of its initial value. These measurements were performed using tobramycin-binding E-AB sensors at controlled 25° C. and under continuous stirring. Shaded areas represent the standard deviation between 5 electrodes.

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and component parts, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Bind: As used herein, “bind,” in the context of pathogen detection, refers to a state in which a first chemical structure (e.g., a therapeutic agent) is sufficiently associated a second chemical structure (e.g., a bioreceptor) such that the association between the first and second chemical structures can be detected.

Detecting: As used herein, “detecting,” “detect,” or “detection” refers to an act of determining the existence or presence of one or more target analytes in a sample.

Biomolecule: As used herein, “biomolecule” refers to an organic molecule produced by a living organism. Examples of biomolecules, include macromolecules, such as nucleic acids, proteins, carbohydrates, and lipids.

Bioreceptor: As used herein, “bioreceptor” refers to a biochemical structure that receives or binds other chemical structures (e.g., therapeutic agents, nucleic acids, proteins, metabolites, and the like).

Sample: As used herein, “sample” means anything capable of being analyzed using a device or system disclosed herein. Exemplary sample types include environmental samples and biological samples. In some embodiments, subjects exhale, spit, sneeze, cough, and/or the like to produce aerosolized samples.

Specifically Bind: As used herein, “specifically bind,” in the context of pathogen detection, refers to a state in which substantially only target chemical structures (e.g., biomolecules) are sufficiently associated with a corresponding or cognate binding agent, to the exclusion of non-target chemical structures, such that the association between the target chemical structures and the binding agent can be detected.

System: As used herein, “system” in the context of analytical instrumentation refers a group of objects and/or devices that form a network for performing a desired objective.

Subject: As used herein, “subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

### DETAILED DESCRIPTION

Electrochemical, aptamer-based (E-AB) sensors support continuous, real-time measurements of specific molecular targets in complex fluids such as undiluted serum, among many other sample types. They typically achieve these measurements by using redox-reporter-modified, electrode-attached aptamers that undergo target binding-induced conformational changes which, in turn, change electron transfer between the reporter and the sensor surface. Traditionally, E-AB sensors are interrogated via pulse voltammetry to monitor binding-induced changes in transfer kinetics. While these pulse techniques are sensitive to changes in electron transfer, they also respond to progressive changes in the sensor surface driven by biofouling or monolayer desorption and, consequently, present significant drift. Moreover, as described herein, it has been empirically observed that differential voltage pulsing can accelerate monolayer desorption from the sensor surface, presumably via field-induced actuation of aptamers. The present disclosure, in contrast, demonstrates the advantages of employing cyclic voltammetry to measure electron transfer changes directly in some embodiments. In certain embodiments, target concentration is reported via changes in the peak-to-peak separation, ΔEP, of cyclic voltammograms. Because the magnitude of ΔEP is insensitive to variations in the number of aptamer probes on the electrode, ΔEP-interrogated E-AB sensors are resistant to drift and show decreased batch-to-batch and day-to-day variability in sensor performance. Moreover, ΔEP-based measurements can also be performed in a few hundred milliseconds and are, thus, competitive with other sub-second interrogation strategies such as chronoamperometry, but with the added benefit of retaining sensor capacitance information that can report on monolayer stability over time.

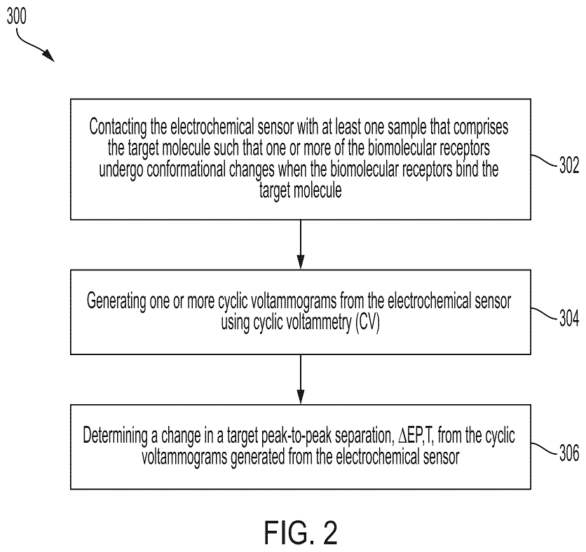

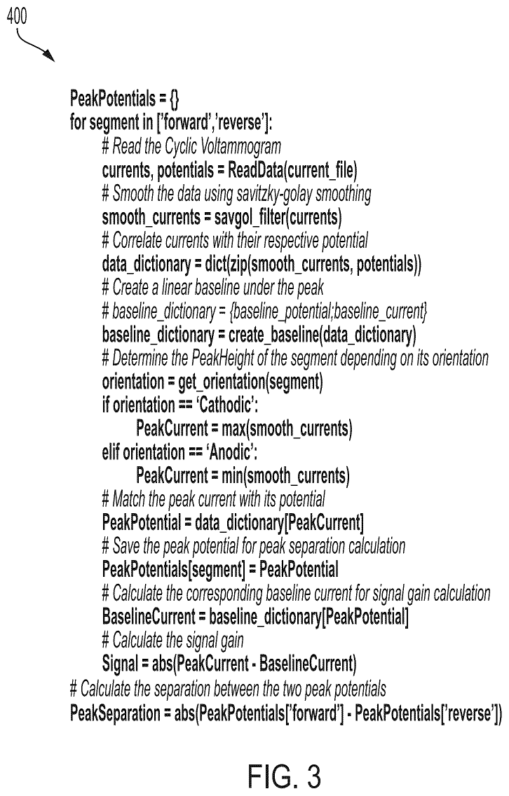

To illustrate some of these aspects, FIG. 2 is a flow chart that schematically depicts steps in a method of detecting a target molecule using an electrochemical sensor comprising biomolecular receptor-bound redox reporters according to an exemplary embodiment. As shown, method 300, which is typically computer-implemented, includes contacting the electrochemical sensor with at least one sample that comprises the target molecule (e.g., a biomolecule or the like) such that one or more of the biomolecular receptors undergo conformational changes when the biomolecular receptors bind the target molecule (step 302). In some embodiments, the biomolecular receptor comprises an aptamer. In some embodiments, the biomolecular receptor comprises a deoxyribonucleic acid (DNA) molecule. In some embodiments, the redox reporters comprise methylene blue (MB). Method 300 also includes generating one or more cyclic voltammograms from the electrochemical sensor using cyclic voltammetry (CV) (step 304). In addition, method 300 also includes determining a change in a target peak-to-peak separation, ΔE_{P,T}, from the cyclic voltammograms generated from the electrochemical sensor to detect the target molecule using the electrochemical sensor (step 306). In some embodiments, the electrochemical sensor comprises a wearable device that is worn by a subject (e.g., integrated into an item of clothing, a watch, or the like). To further illustrate, FIG. 3 shows algorithm 400 that can be used to implement aspects of method 300 according to an exemplary embodiment.

In some embodiments, method 300 includes comparing the ΔE_{P,T} to a no target peak-to-peak separation, ΔE_{P,NT}, determined from one or more cyclic voltammograms generated from the electrochemical sensor in the absence of the target molecule. In some embodiments, method 300 includes determining a concentration of the target molecule in the sample by comparing the ΔE_{P,T} to a standard curve. In some embodiments, method 300 includes determining the ΔE_{P,T} from at least a first cyclic voltammogram and at least a second cyclic voltammogram generated from the electrochemical sensor. In some embodiments, method 300 includes correlating at least two currents with corresponding peak potentials and calculating a separation between the peak potentials.

In some embodiments, method 300 includes determining a concentration of the target molecule in the sample via the change in the target peak-to-peak separation, ΔE_{P,T}. In some embodiments, the electrochemical sensor is substantially resistant to drift. In some embodiments, method 300 includes determining the change in the target peak-to-peak separation, ΔE_{P,T}, from the cyclic voltammograms with about 900 milliseconds, about 800 milliseconds, about 700 milliseconds, about 600 milliseconds, about 500 milliseconds, about 400 milliseconds, about 300 milliseconds, about 200 milliseconds, about 100 milliseconds, or less of contacting the electrochemical sensor with the sample. In some embodiments, method 300 includes generating the cyclic voltammograms from the electrochemical sensor using a voltage scanning rate of about 5 V s⁻¹ or more. In some embodiments, the voltage scanning rate is between about 5 V s⁻¹ and about 10 V s⁻¹. In some embodiments, method 300 includes continuously monitoring the change in the target peak-to-peak separation, ΔE_{P,T} over time from multiple cyclic voltammograms generated from the electrochemical sensor.

In some embodiments, the sample is substantially unprocessed. In some embodiments, the sample comprises an environmental sample. In some embodiments, the target molecule comprises a therapeutic agent. In some embodiments, the methods disclosed herein include generating a dose-response curve for the therapeutic agent. In some embodiments, the target molecule comprises a metabolite. In some embodiments, the target molecule comprises a biomolecule. In some embodiments, the sample comprises a biological sample. In some embodiments, the biological sample is obtained from a subject. In some embodiments, the biological sample is selected from the group consisting of, for example, serum, plasma, blood, urine, feces, semen, and cerebrospinal fluid, among other sample types.

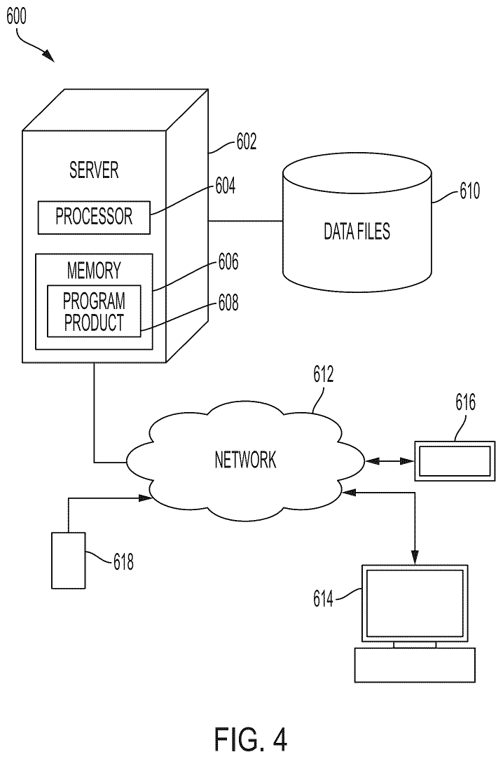

The present disclosure also provides various systems and computer program products or machine-readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 4 provides a schematic diagram of an exemplary system suitable for use with implementing at least aspects of the methods disclosed in this application. As shown, system 600 includes at least one controller or computer, e.g., server 602 (e.g., a search engine server), which includes processor 604 and memory, storage device, or memory component 606, and one or more other communication devices 614, 616, (e.g., client-side computer terminals, telephones, tablets, laptops, other mobile devices, etc. (e.g., for receiving data for further analysis, etc.)) positioned remote from electrochemical sensor 618, and in communication with the remote server 602, through electronic communication network 612, such as the Internet or other internetwork. Communication devices 614, 616 typically include an electronic display (e.g., an internet enabled computer or the like) in communication with, e.g., server 602 computer over network 612 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 600 also includes program product 608 stored on a computer or machine readable medium, such as, for example, one or more of various types of memory, such as memory 606 of server 602, that is readable by the server 602, to facilitate, for example, a guided search application or other executable by one or more other communication devices, such as 614 (schematically shown as a desktop or personal computer). In some aspects, system 600 optionally also includes at least one database server, such as, for example, server 610 associated with an online website having data stored thereon searchable either directly or through search engine server 602. System 600 optionally also includes one or more other servers positioned remotely from server 602, each of which are optionally associated with one or more database servers 610 located remotely or located local to each of the other servers. The other servers can beneficially provide service to geographically remote users and enhance geographically distributed operations.

As understood by those of ordinary skill in the art, memory 606 of the server 602 optionally includes volatile and/or nonvolatile memory including, for example, RAM, ROM, and magnetic or optical disks, among others. It is also understood by those of ordinary skill in the art that although illustrated as a single server, the illustrated configuration of server 602 is given only by way of example and that other types of servers or computers configured according to various other methodologies or architectures can also be used. Server 602 shown schematically in FIG. 4, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 600. As also understood by those of ordinary skill in the art, other user communication devices 614, 616 in these aspects, for example, can be a laptop, desktop, tablet, personal digital assistant (PDA), cell phone, server, or other types of computers. As known and understood by those of ordinary skill in the art, network 612 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 608 is optionally in the form of microcode, programs, cloud computing format, routines, and/or symbolic languages that provide one or more sets of ordered operations that control the functioning of the hardware and direct its operation. Program product 608, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 608 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 608 is optionally copied from the computer-readable medium to a hard disk or a similar intermediate storage medium. When program product 608, or portions thereof, are to be run, it is optionally loaded from their distribution medium, their intermediate storage medium, or the like into the execution memory of one or more computers, configuring the computer(s) to act in accordance with the functionality or method of various aspects. All such operations are well known to those of ordinary skill in the art of, for example, computer systems.

To further illustrate, in certain aspects, this application provides systems that include one or more processors, and one or more memory components in communication with the processor. The memory component typically includes one or more instructions that, when executed, cause the processor to provide information that causes at least one result, data, and/or the like to be displayed or otherwise indicated (e.g., via a result indicator of electrochemical sensor 618 and/or via communication devices 614, 616 or the like) and/or receive information from other system components and/or from a system user (e.g., via communication devices 614, 616, or the like).

In some aspects, program product 608 includes non-transitory computer-executable instructions which, when executed by electronic processor 604 perform at least: generating one or more cyclic voltammograms from the electrochemical sensor using cyclic voltammetry (CV) when the electrochemical sensor is contacted with at least one sample that comprises the target molecule such that one or more of the biomolecular receptors undergo conformational changes when the biomolecular receptors bind the target molecule; and determining a change in a target peak-to-peak separation, ΔE_{P,T}, from the cyclic voltammograms generated from the electrochemical sensor to detect the target molecule in the sample.

Additional exemplary aspects of the present disclosure also shown in the following slides:

### Example

### Interrogation of Electrochemical Aptamer-Based Sensors Via Peak-to-Peak Separation in Cyclic Voltammetry Improves Temporal Stability and Batch-to-Batch Variability in Biological Fluids

### Results and Discussion

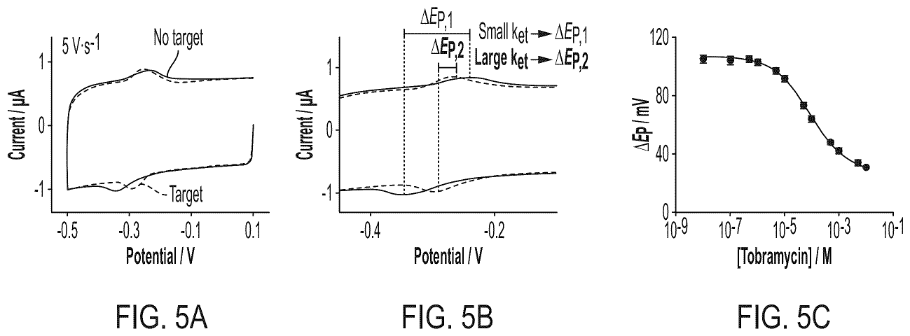

The magnitude of ΔE_{P} in cyclic voltammetry can be used to interrogate changes in the apparent electron transfer kinetics of E-AB sensors. ΔE_{P} is affected by a mass transfer (diffusional) component related to the conformation switching behavior of aptamers; thus, measured changes in ΔE_{P} reflect target-binding induced changes in aptamer conformation. We first illustrate this effect employing tobramycin-detecting E-AB sensors (FIG. 5), which have been extensively validated in the literature and represent a well-understood model system. In these E-AB sensors, interrogation by cyclic voltammetry in the absence of target and at voltage scanning rates of ˜1-10 V·s⁻¹ results in voltammograms with a ΔE_{P} ˜100 mV (FIG. 5A). However, the addition of a saturating concentration of tobramycin (10 mM) causes a significant increase in apparent electron transfer kinetics with a concomitant decrease in ΔE_{P} to ˜30 mV (FIG. 5A, B). Similar measurements at varying tobramycin concentrations can be translated into dose-response curves (FIG. 5C). The dissociation constant estimated by extrapolating the mid-point of these dose-response curves to the x-axis, K_{D}˜80 μM, is similar to previously reported K_{D} measurements based on chronoamperometric and differential pulse measurements.

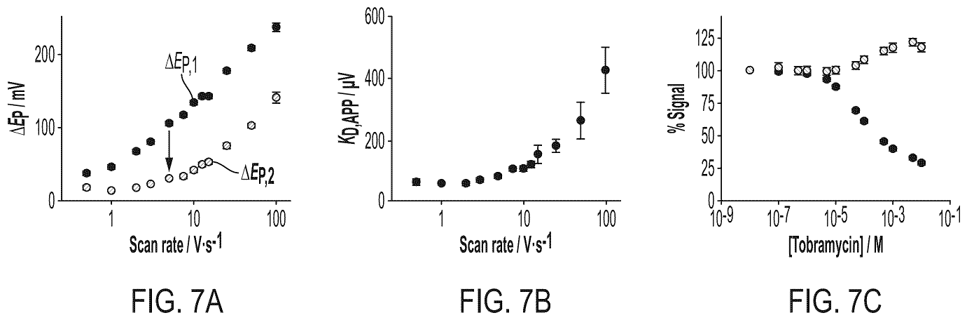

The magnitude of ΔE_{P} in cyclic voltammograms of E-AB sensors can be a strong function of the voltage scanning rate. To investigate this effect, we interrogated a fresh batch of tobramycin-detecting E-AB sensors at scanning rates ranging from 0.5 to 100 V·s⁻¹, in the presence and absence of saturating tobramycin concentrations (FIG. 7A). At scanning rates <0.5 V·s⁻¹, cyclic voltammograms presented a ΔE_{P} that closely approached 0 mV, as expected for an electrode-bound, quasi-reversible system such as the one considered here. However, at faster scanning rates the magnitude of ΔE_{P} progressively increased both in the presence or absence of tobramycin, albeit not with the same correlation function. This increase in ΔE_{P} occurred because the CV scanning rates became faster than the diffusional contribution arising from the conformation-switching behavior of aptamers (i.e., the measurement rate became faster than the apparent electron transfer rate). Yet, because the addition of target moves the equilibrium in

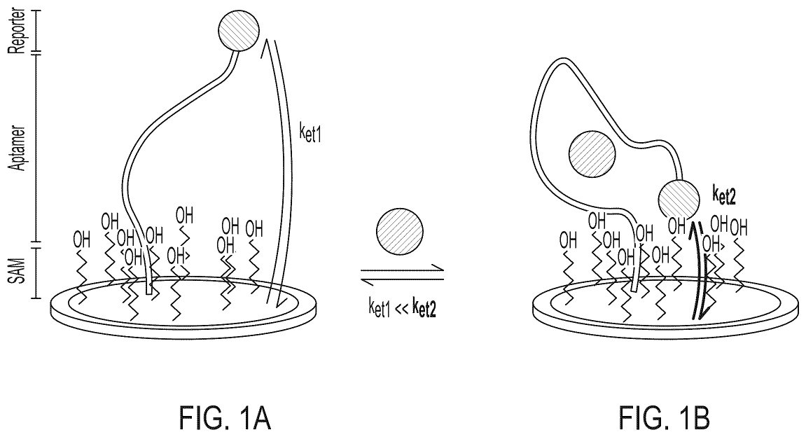

FIG. 1 to the right (i.e., it accelerates electron transfer), ΔE_{P,2} is always smaller than ΔE_{P,1}. Thus, the magnitude of ΔE_{P} can be effectively used to interrogate the electron transfer state of tobramycin-detecting E-AB sensors, with maximum signal change observed at scanning rates between 5 and 10 V·s⁻¹.

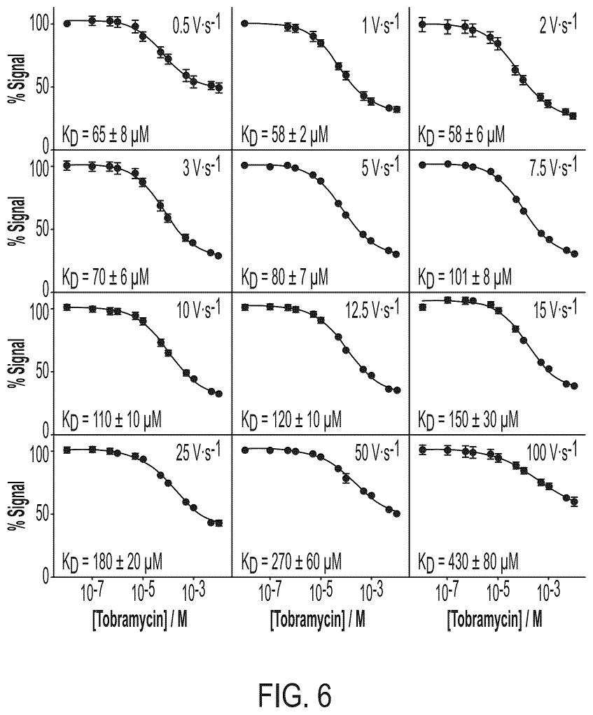

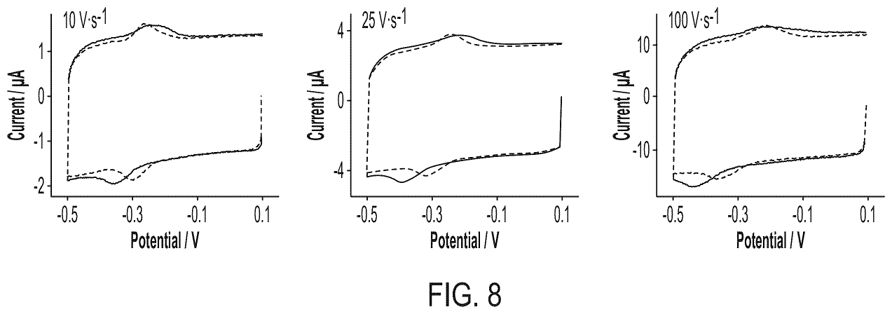

As with all E-AB interrogation methods, there is a tradeoff between the maximum signal gain observed by ΔE_{P} and the apparent dissociation constant of the sensors. To illustrate this, we measured dose-response curves as shown in FIG. 5C at scanning rates ranging from 0.5 to 100 V·s⁻¹ (FIG. 6). Then, we determined the apparent dissociation constant at each scanning rate by performing a non-linear regression of the data sets using the Hill isotherm. The resulting graph of K_{D} vs voltage scanning rate (FIG. 7B) shows a strong dependence on scanning rate, with a significant deviation from published K_{D}s (determined by square-wave voltammetry or chronoamperometry) at scanning rates >10 V·s⁻¹. Moreover, the shape of the voltammograms was distorted at fast scanning rates, preventing the accurate determination of ΔE_{P} (FIG. 8). Thus, taking into consideration the results presented in FIGS. 7A and B, we determined that the best CV interrogation rate for tobramycin-detecting E-AB sensor was 5 V·s⁻¹.

E-AB interrogation via ΔE_{P} achieves larger signal gains relative to interrogation based on CV peak heights. To illustrate this point, we measured dose-response curves at the optimal voltage scanning rate of 5 V·s⁻¹ and compared the response based on voltammogram peak currents, I_{P}, at varying tobramycin concentrations relative to the proposed ΔE_{P}-based method (FIG. 7C). To achieve a direct comparison between approaches, we calculated the relative change in signal at each concentration with respect to the signal measured in the absence of target (this is common practice in the field). By graphing I_{P} vs concentration, we observed a ‘signal-on’ type of response, with I_{P} increasing to a plateau of 20% relative signal change at saturation. Using ΔE_{P}, in contrast, we observed a ‘signal-off’ type of response, with ΔE_{P} decreasing by 80% relative signal change at saturation. From these results we conclude that, on the one hand, ΔE_{P} achieves a larger signal gain (i.e., larger net signal change), thereby improving the sensitivity of CV-interrogated E-ABs. On the other hand, ΔE_{P} provides a signal-off response that is less desirable than the signal-on response achieved by monitoring I_{P}. Typically, the use of signal-off sensing is sub-optimal for continuous monitoring in complex fluids, as matrix effects can induce signal decreases unrelated to fluctuations in target concentration that can be confounding during sensing. However, below we demonstrate that the ΔE_{P}-based interrogation of E-ABs achieves, in fact, superior stability than I_{P}-based interrogation methods with less batch-to-batch measurement variability in, for example, undiluted serum.

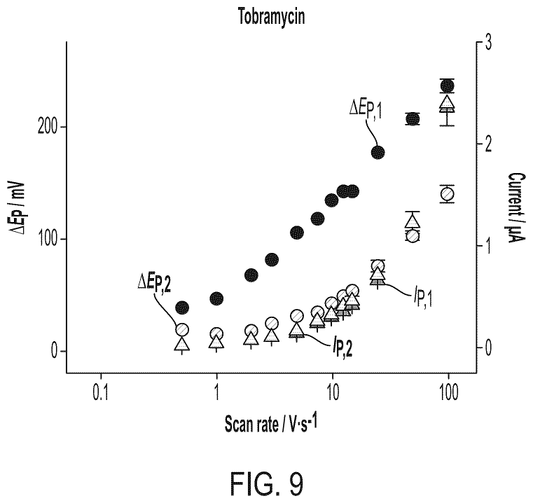

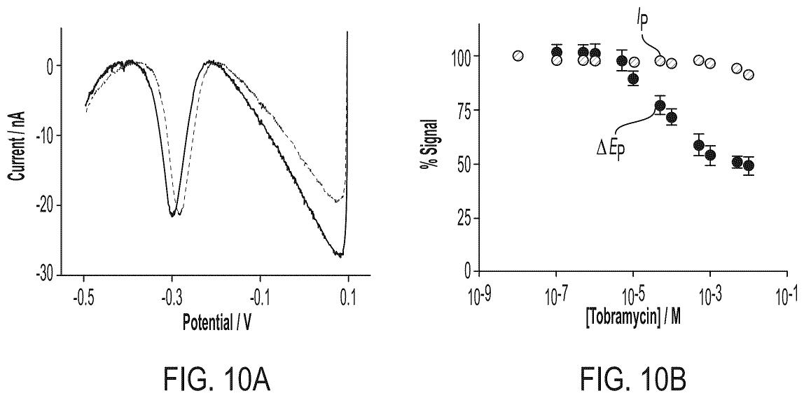

The enhanced signal gain seen with ΔE_{P}-based interrogation can be explained by its strong correlation to electron transfer kinetics. For diffusion-less, quasi-reversible systems, Laviron established that ΔE_{P} can be accurately used to determine electron transfer rate constants (Laviron, E. J. Electroanal. Chem. Interf. Electrochem. 1979, 101, 19). Although, technically, the E-AB system is not entirely diffusion-less—previous works have demonstrated a diffusional contribution to the electrochemical response for DNA sequences longer that 10 nucleotides—ΔE_{P} does directly reflect binding-induced changes in apparent electron transfer kinetics. Voltammetric peak currents, in contrast, are more reflective of the total number of electrons being transferred in the voltammetric sweep (i.e., total moles of reporter-modified aptamer), and not of electron transfer rate. We experimentally demonstrate this for tobramycin-binding E-AB sensors by comparing side-by-side the behavior of ΔE_{P} and I_{P} in the absence and presence of target and at various scanning rates (FIG. 9). This analysis shows that, at the optimal voltage scanning rate of 5 V·s⁻¹, challenging the E-AB sensors with target causes a more pronounced change in ΔE_{P} than in I_{P}. This effect is perhaps most emphasized at scanning rates <1V·s⁻¹, where I_{P} is completely insensitive to target additions (FIG. 10).

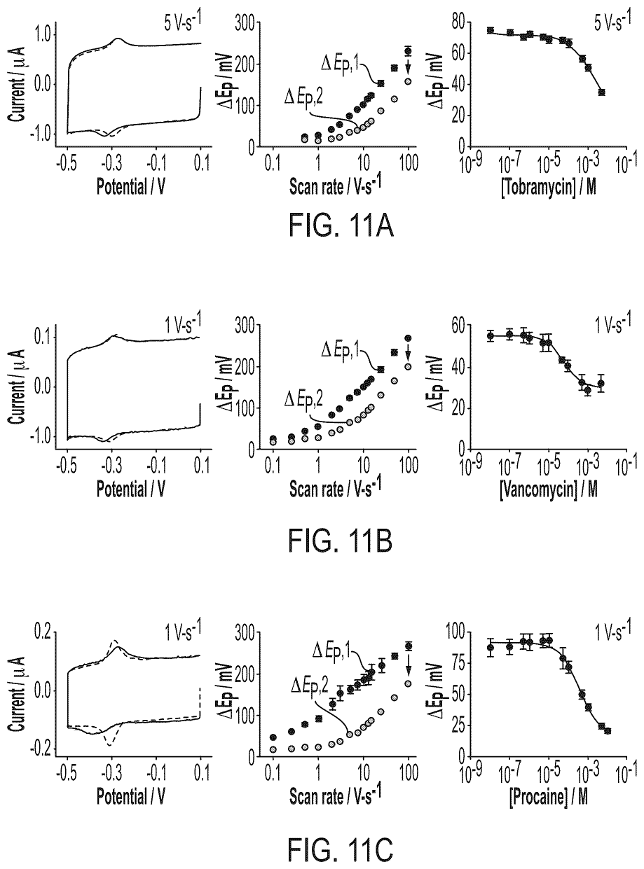

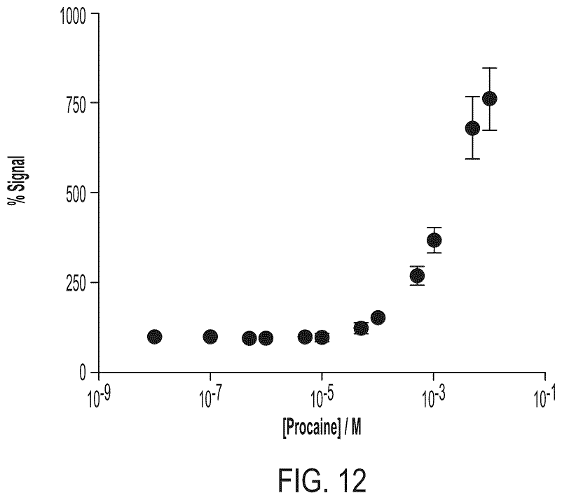

ΔE_{P}-based interrogation of E-AB sensors supports molecular measurements in complex media such as undiluted serum. Moreover, the approach can be used to interrogate E-AB sensors detecting different molecular targets. We demonstrate these points here by interrogating sensors employing aptamers that bind to 3 structurally different therapeutics: the aminoglycoside tobramycin, the glycopeptide vancomycin, and the amino ester procaine (FIG. 11). Using ΔE_{P}-based interrogation in undiluted serum, and mapping CV scan rates to identify the voltage sweep speed producing maximum change in ΔE_{P}, we observed that tobramycin detecting E-AB sensors (FIG. 11A) present a significant decrease in affinity relative to measurements performed in phosphate-buffered saline (FIG. 7C). Furthermore, the relative change in ΔE_{P} at 10 mM tobramycin was 30% less than the gain seen in phosphate-buffered saline. Both of these effects were previously reported for serum tobramycin E-AB measurements performed by chronoamperometry, confirming that ΔE_{P} is not more prone to fouling than, for example, chronoamperometric current decay lifetimes. Similarly, interrogating vancomycin-detecting sensors using ΔE_{P} (FIG. 11B) resulted in serum calibration curves that did not differ in affinity relative to previous measurements performed in blood; i.e., the K_{D} remained at ˜60 μM, with a total relative change in ΔE_{P} at saturating vancomycin concentrations of ˜50%. Last, procaine-detecting E-AB sensors presented the largest change in ΔE_{P}, corresponding to a relative decrease of ˜80% at saturating procaine concentrations (FIG. 11C). Based on our calibration curve, the procaine-binding aptamer has a K_{D} of ˜500 μM, slightly better than the affinity obtained by, for example, square-wave voltammetry (K_{D}˜2 mM, FIG. 12).

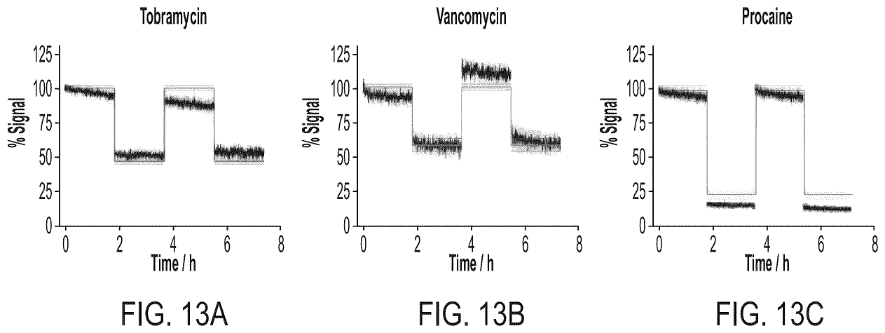

Because ΔE_{P} can rapidly respond to fluctuating target levels, our approach supports continuous molecular monitoring in complex fluids. We evaluate this by serially interrogating E-AB sensors by CV in undiluted serum in the presence and absence of saturating target concentrations (FIG. 13). To achieve real-time visualization of these measurements, we adapted SACMES30—an open-source, Python-based script previously reported by our group— to the real-time processing of ΔE_{P}. This script continuously checks for the presence of new CV files in a computer folder, is able to calculate ΔE_{P} for any new file within milliseconds, and then graphs the data, thereby supporting real-time visualization (see Methods section for more details). To estimate the percentage contribution of sensor drift to our measurements, we calculated the signal change at every time point relative to the first measurement in each time course. The resulting graphs (FIG. 13) show that, in the absence of target, the three sensors considered display a continuous and slow (˜0.08%.min⁻¹) decrease in the peak-to-peak separation (˜10% in 2 hours). We attribute this drift to the progressive, non-specific adsorption of serum proteins to the electrode surface, which can bring the redox reporter closer to the electrode surface thereby increasing electron transfer kinetics (and consequently decreasing ΔE_{P}). The addition of target, in contrast, caused a sharp increase in electron transfer rate that led to a steep drop in ΔE_{P}. For reasons we do not yet understand, E-AB signals in the bound state seem more stable in all cases, with drift rates approximately one order of magnitude below those seen in the absence of target. Our results also demonstrate the reversibility of ΔE_{P}, which achieves almost full recovery of baseline signal (within 15% error) after a serum wash.

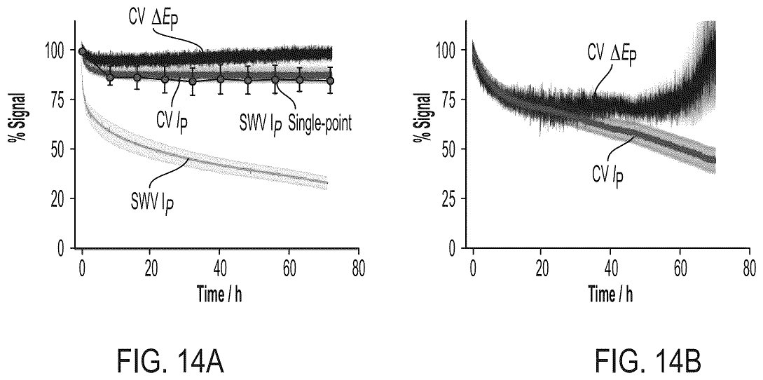

A critical point to consider when selecting the most suitable electrochemical interrogation technique for E-AB sensors is how the voltage program of that technique can affect long-term sensor stability. The added value of CV over, for example, square-wave voltammetry, is that its linear voltage sweeps can be gentler on biosensor interfaces than differential voltage pulsing. To illustrate this beneficial effect, we compared the signal stability of tobramycin-detecting E-ABs under continuous electrochemical interrogation when using square-wave voltammetry vs CV (FIG. 14A). First, we observed that sensors that were serially interrogated by square-wave voltammetry in phosphate-buffered saline, every 5 s for 72 hours (50,000 total measurements), underwent a 50% drop in peak current within the first 10 hours (red trace in FIG. 14A), followed by a more moderate but still progressive decrease (˜0.4%·h⁻¹) down to only ˜35% of the initial current. This degradation was completely eliminated when we repeated the experiment using a measurement frequency of one square-wave voltammogram every 8 hours (10 total measurements, green circles in FIG. 14A), a clear indication that measurement frequency contributes to E-AB signal decay when using square-wave voltammetry. Sensors serially interrogated by CV every 5 s (70,000 scans), in contrast, only showed a modest initial decrease in signal (˜10% in either I_{P} or ΔE_{P}, blue and black traces in FIG. 14A, respectively) over the first five hours, which we attribute to the slow rearrangement or desorption of monolayer groups. We speculate the rapid, bidirectional voltage fluctuations occurring during square-wave interrogation cause field-induced oscillations of electrode-bound DNA aptamers that accelerate DNA desorption from the electrode surface. Our group has previously demonstrated that monolayer desorption plays a critical role in E-AB signal drift; thus, it is conceivable that interrogation approaches that heavily drive bidirectional DNA actuation may further contribute to E-AB signal decay over time.

The similar drift resistance of CV I_{P} and ΔE_{P} seen in phosphate-buffered saline does not translate to unprocessed biological fluids, where ΔE_{P} outperforms I_{P} in long-term stability. This difference is revealed by repeating the CV experiment from FIG. 14A in undiluted human serum (FIG. 14B). Here, the initial decay accounts for ˜25% signal loss during the first 10 hours. We attribute this signal decrease, both in ΔE_{P} and I_{P}, to a combination of progressive monolayer desorption (both the blocking alkanethiols and aptamers), and non-specific adsorption of serum proteins. Protein adsorption sterically pushes aptamer molecules closer to the electrode surface increasing E-AB electron transfer kinetics, which we see in our measurements as a progressive decrease in ΔE_{P}. However, following this initial and rapid decay, I_{P} continues to linearly drop (˜0.5%·h⁻¹) as monolayer elements continue to progressively desorb from the sensor surface, down to ˜45% of the initial current at 60 h. ΔE_{P} measurements, in contrast, are less sensitive to monolayer desorption, presenting a stable response (˜75% of initial value) for 50 hours following the first decay. This drift resistance remains until aptamer desorption is such that our data processing software becomes unable to find current peaks in the voltammograms. At that point, roughly 60 h after the beginning of the experiment, monolayer desorption is such that our software only visualizes the increasing capacitive current of the voltammograms.

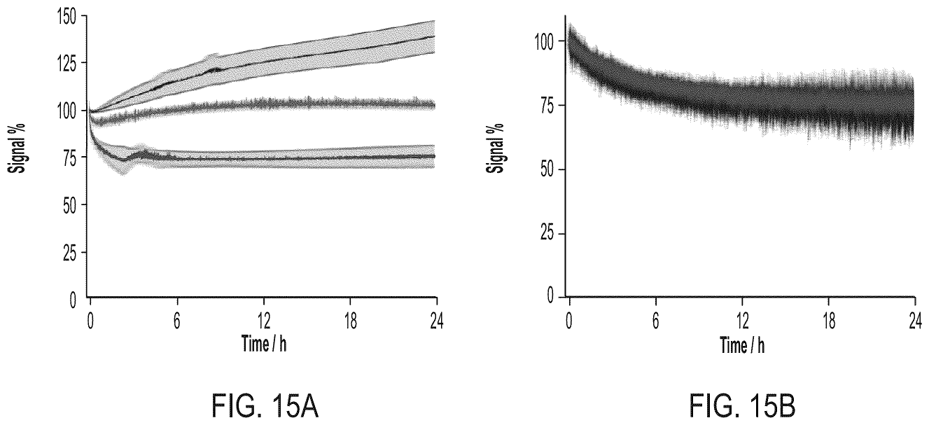

One important advantage of ΔE_{P}-based E-AB interrogation over square-wave voltammetry is that ΔE_{P} presents less batch-to-batch and day-to-day variations in sensing performance in unprocessed serum. To illustrate this effect, we serially interrogated three independent batches of six E-AB sensors each by square-wave voltammetry (at the same square-wave frequency and amplitude) in undiluted serum, every 5 s for 24 h on separate days, observing significant variability in peak currents (FIG. 15A). Specifically, in the first three hours of continuous measurements, one batch presented an initial signal decay of ˜10% (green line) vs ˜25% (blue line) for a second, while a third presented a current gain (gray line). Moreover, after the first three hours the currents remained stable for one batch (blue line) but increased for the other two (green and gray lines). In contrast, interrogating three new batches using ΔE_{P} and a similar measurement rate, we observed identical signal behavior across batches and between days. For all three experimental repeats, we observed an initial decrease in the first few hours (as in FIG. 14B), followed by a stable readout for about 18 hours. These differences in baseline performance between techniques arise from the sensitivity of each technique toward changes in the sensing layer. While square-wave voltammetry responds to both aptamer desorption from the sensor surface and non-specific protein adsorption, ΔE_{P}-based interrogation is only sensitive to changes in electron transfer from the redox reporter and, therefore, only drifts with the sensor interface rearrangement seen during the first few hours of interrogation.

The work presented here is focused on the use of ΔE_{P} to interrogate E-AB sensors for the detection of small-molecule targets in biological media. However, we note the same interrogation approach can be readily applied to DNA-based sensors used in the detection of other target types and in different media, as long as the sensing mechanism involves binding-induced changes in reporter electron transfer kinetics. For example, previous works have shown changes in ΔE_{P} upon hybridization of surface-bound, MB-modified DNA with complementary strands. In these cases, DNA hybridization moves MB further away from the electrode surface, leading to an increase in ΔE_{P} (i.e. signal-ON sensors). Similarly, the approach can be used to interrogate E-DNA sensors containing an antibody-binding epitope, aptamer-based sensors binding to protein targets, or DNA-origami based sensors binding to single-entity, mesoscale targets.

### CONCLUSIONS

Method selection for the interrogation of E-AB sensors typically requires careful consideration of the intended application, the time resolution needed, and the importance given to electrochemical information offered by different methods. We have previously discussed the advantages and disadvantages of many electrochemical methods, mentioning in passing that cyclic voltammetry is most often used for sensor characterization and not for interrogation. Here, however, we describe an approach to use cyclic voltammetry for the direct interrogation of E-AB sensors, with the added benefit of retaining the rich electrochemical information provided by this technique. We have also adapted SACMES, a software we previously reported, to enable real-time, CV-based interrogation of E-AB sensors. The new version of the software allows tracking of voltammetric peak currents and voltages, ΔE_{P}, and arbitrary currents at user specified voltages, continuously and with millisecond processing speeds. Using this platform, we demonstrate that the peak-to-peak separation in cyclic voltammograms of E-AB sensors can be used to directly, rapidly and reversibly interrogate this class of sensors. The method works across sensors binding to structurally different molecular targets and in complex media such as undiluted whole serum. Moreover, because ΔE_{P} is not a function of the moles of aptamer bound to the electrode surface but of the fractional populations of bound vs unbound aptamers, this parameter is drift resistant and expands the operational lifetime of E-AB sensors relative to interrogation based on voltammetric peak currents. This approach supports fast, sub-second voltammetric measurements that could be valuable for the study of aptamer-target binding and dissociation kinetics, or for the time resolved study of dynamic processes in biological systems.

### Methods

Chemicals and materials. Phosphate buffered saline (PBS; 11.9 mM HPO32-, 137 mM NaCl, 2.7 mM KCl; pH=7.4), sulfuric acid, sodium hydroxide, and procaine hydrochloride were purchased from Fisher Scientific (Waltham, MA). 6-mercaptohexanol, tris(2-carboxyethyl)phosphine hydrochloride (TCEP), and the three nucleic acid aptamers were purchased from Sigma-Aldrich (St. Louis, MO). Human serum was purchased from BioIVT (Washington, D.C.). Tobramycin sulfate was purchased from GoldBio (St. Louis, MO). Vancomycin hydrochloride was purchased from Alfa-Aesar (Ward Hill, MA). All solutions were prepared using ultrapure Milli-Q water with 18 Ω resistance.

The nucleic acid aptamer sequences used in this work were obtained from previous works:

| | | | SEQ |
|---|---|---|---|
| | | | ID |
| Target | Sequence | Ref | NO. |
| Tobramycin | 5′-GGGACTTGGTTTAGGTAATGAGT | 3 | 1 |
| | CCC-3′ | | |
| Vancomycin | 5′-CGAGGGTACCGCAATAGTACTTA | 28 | 2 |
| | TTGTTCGCCTATTGTGGGTCGG-3′ | | |
| Procaine | 5′-GACAAGGAAATCCTTCAACGAAG | 17 | 3 |
| | TGGGTC-3′ | | |

These sequences were purchased modified on the 5′end with hexanethiol and on the 3′end with MB, and double HPLC purified. To prepare aptamer solutions for electrode modification, 1 μL of 100 μM of the modified sequences was incubated with 2 μL of 5 mM TCEP solution in water for 1 hour to reduce the disulfide bonds. Then, we diluted the aptamer solutions in 1 mM 6-mercaptohexanol aqueous solution to a final aptamer concentration of 500 nM.

Electrochemical measurements. Gold working (PN 002314, d=1.6 mm) and coiled platinum wire counter electrodes (PN 012961) were purchased from ALS Inc. (Japan). Ag/AgCl (1M KCl) reference electrodes (PN CHI111) were purchased from CH Instruments (USA). All the electrochemical measurements were done using a multichannel potentiostat CHI 1040C (CH Instruments). Cyclic voltammograms were recorded at different scan rates in the potential window from −0.5 V to +0.1 V vs Ag/AgCl (1M KCl). Square-wave voltammograms were recorded from 0 V to −0.5 V vs Ag/AgCl (1M KCl) with an amplitude of 50 mV, a step size of 1 mV, and at 300 Hz. When specified, the temperature of the electrochemical cell was held constant by using a Huber Microprocessor Control water recirculation bath obtained from Huber (USA).

Electrode modification. Gold electrodes were polished on a 1200/P2500 silicon carbide grinding paper (PN 36-08-1200, Buehler, USA), and on a cloth pad and alumina slurry (PN CF-1050, BASi, USA). After rinsing and sonicating them with water for 1 min to remove polishing debris, the were electrochemically activated by continuous cycles of CV from −0.3 V to −1.6 V in 0.5 M NaOH, and from 0 V to 1.6 V in 0.5 M H₂SO₄, 250 times in each solution at a scan rate of 0.5 V/s. After this, electrodes were rinsed with water and dipped in the aptamer-thiol mixture for 15 h at room temperature. After rinsing with water, electrodes were ready for use. For the long-term stability experiments of FIGS. 14 and 15, we used a backfilling protocol to modify the electrodes. Briefly, we incubated the activated gold electrodes in 500 nM solutions of the MB-modified aptamer for 2 hours. After rinsing in deionized water, electrodes were dipped in 1 mM solution of 6-mercaptohexanol prepared in water for 15 hours at room temperature. This backfilling protocol allowed us to slightly increase the number of aptamer molecules immobilized on the electrode surface and obtain larger voltametric peaks.

Data analysis. To process the data obtained from the stability experiments where the sensors were continuously interrogated for long periods of time, we used a previously reported, open-source Python script called SACMES.30 This software supports the real-time analysis, visualization, and control of electrochemical data with millisecond resolution, providing users the ability to extract peak currents (SWV, CV), peak-to-peak separation (CV), half-lives (Chronoamperometry), and area under the curve (SWV, CV). Furthermore, this software provides the user with dynamic control over smoothing and regression algorithms used to filter and fit the raw data.

The data obtained from all experiments were processed using data analysis software Origin Pro v8.5. Multi-panel figures were assembled using Adobe Creative Cloud 2021.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, devices, systems, computer readable media, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

1. A method of detecting a target molecule using an electrochemical sensor comprising biomolecular receptor-bound redox reporters, the method comprising:
contacting the electrochemical sensor with at least one sample that comprises the target molecule such that one or more of the biomolecular receptors undergo conformational changes when the biomolecular receptors bind the target molecule;
generating one or more cyclic voltammograms from the electrochemical sensor using cyclic voltammetry (CV); and,
determining a change in a target peak-to-peak separation, ΔE_{P,T}, from the cyclic voltammograms generated from the electrochemical sensor, thereby detecting the target molecule using the electrochemical sensor.

2. The method of claim 1, wherein:
the determining step comprises comparing the ΔE_{P,T} to a no target peak-to-peak separation, ΔE_{P,NT}, determined from one or more cyclic voltammograms generated from the electrochemical sensor in the absence of the target molecule; or,
the determining step comprises correlating at least two currents with corresponding peak Potentials and calculating a separation between the peak potentials.

3. The method of claim 1, comprising:
determining a concentration of the target molecule in the sample by comparing the ΔE_{P,T} to a standard curve;
determining the ΔE_{P,T} from at least a first cyclic voltammogram and at least a second cyclic voltammogram generated from the electrochemical sensor;
determining a concentration of the target molecule in the sample via the change in the target peak-to-peak separation, ΔE_{P,T}; or,
determining the change in the target peak-to-peak separation, ΔE_{P,T}, from the cyclic voltammograms with about 900 milliseconds, about 800 milliseconds, about 700 milliseconds, about 600 milliseconds, about 500 milliseconds, about 400 milliseconds, about 300 milliseconds, about 200 milliseconds, about 100 milliseconds, or less of contacting the electrochemical sensor with the sample.

4. (canceled)

5. (canceled)

6. The method of claim 1, wherein:
the biomolecular receptor comprises an aptamer; or,
the biomolecular receptor comprises a deoxyribonucleic acid (DNA) molecule.

7. (canceled)

8. The method of claim 1, wherein the redox reporters comprise methylene blue (MB).

9. (canceled)

10. The method of claim 1, wherein the electrochemical sensor is substantially resistant to drift.

11. (canceled)

12. The method of claim 1, comprising generating the cyclic voltammograms from the electrochemical sensor using a voltage scanning rate of about 5 V s⁻¹ or more.

13. The method of claim 12, wherein the voltage scanning rate is between about 5 V s⁻¹ and about 10 V s⁻¹.

14. The method of claim 1, wherein:
the sample is substantially unprocessed; or,
the sample comprises an environmental sample.

15. (canceled)

16. The method of claim 1, wherein:
the target molecule comprises a therapeutic agent
the target molecule comprises a metabolite; or,
the target molecule comprises a biomolecule.

17.-19. (canceled)

20. The method of claim 1, comprising continuously monitoring the change in the target peak-to-peak separation, ΔE_{P,T} over time from multiple cyclic voltammograms generated from the electrochemical sensor.

21.-23. (canceled)

24. The method of claim 1, wherein the electrochemical sensor comprises a wearable device that is worn by the subject.

25. A system, comprising:
at least one electrochemical sensor comprising biomolecular receptor-bound redox reporters; and,
at least one controller operably connected to the electrochemical sensor, which controller comprises, or is capable of accessing, computer readable media comprising non-transitory computer executable instructions which, when executed by at least one electronic processor, perform at least:
generating one or more cyclic voltammograms from the electrochemical sensor using cyclic voltammetry (CV) when the electrochemical sensor is contacted with at least one sample that comprises the target molecule such that one or more of the biomolecular receptors undergo conformational changes when the biomolecular receptors bind the target molecule; and
determining a change in a target peak-to-peak separation, ΔE_{P,T}, from the cyclic voltammograms generated from the electrochemical sensor to detect the target molecule in the sample.

26. A computer readable media comprising non-transitory computer executable instructions which, when executed by at least electronic processor, perform at least:
generating one or more cyclic voltammograms from an electrochemical sensor comprising biomolecular receptor-bound redox reporters using cyclic voltammetry (CV) when the electrochemical sensor is contacted with at least one sample that comprises the target molecule such that one or more of the biomolecular receptors undergo conformational changes when the biomolecular receptors bind the target molecule; and,
determining a change in a target peak-to-peak separation, ΔE_{P,T}, from the cyclic voltammograms generated from the electrochemical sensor to detect the target molecule in the sample.

27. The system of claim 25, wherein the instructions further perform at least:
comparing the ΔE_{P,T} to a no target peak-to-peak separation, ΔE_{P,NT}, determined from one or more cyclic voltammograms generated from the electrochemical sensor in the absence of the target molecule.

28. The system of claim 25, wherein the instructions further perform at least:
determining a concentration of the target molecule in the sample by comparing the ΔE_{P,T} to a standard curve;
determining the ΔE_{P,T} from at least a first cyclic voltammogram and at least a second cyclic voltammogram generated from the electrochemical sensor; or,
determining a concentration of the target molecule in the sample via the change in the target peak-to-peak separation, ΔE_{P,T}.

29.-34. (canceled)

35. The system of claim 25, wherein the cyclic voltammograms are determined from the electrochemical sensor using a voltage scanning rate of about 5 V s⁻¹ or more.

36. The system of claim 25, wherein the target molecule comprises a therapeutic agent and wherein the instructions further perform at least:
generating a dose-response curve for the therapeutic agent.

37. The system of claim 25, wherein the instructions further perform at least:
continuously monitoring the change in the target peak-to-peak separation, ΔE_{P,T} over time from multiple cyclic voltammograms generated from the electrochemical sensor.

38. The system of claim 25, wherein the electrochemical sensor comprises a wearable device that is worn by the subject.
